(19) United States
(12) Patent Application Publication
(10) Pub. No.: US 2010/0267817 A1
(43) Pub. Date: Oct. 21, 2010

(54) LYOPHILIZED PHARMACEUTICAL COMPOSITION WITH IMPROVED RECONSTITUTION TIME CONTAINING TAXANE DERIVATIVES AND METHOD OF MANUFACTURING THE SAME

(76) Inventors: Woo Jae Jang, Gyeonggi-do (KR); Joon-Gyo Oh, Gyeonggi-do (JP); Young Youn Hwang, Gyeonggi-do (JP); Won Jae Choi, Seoul (KR); Key An Um, Gyeonggi-do (KR)

Correspondence Address: FROMMER LAWRENCE & HAUG, 745 FIFTH AVENUE- 10TH FL., NEW YORK, NY, 10151, US

(21) Appl. No.: 12/744,151

(22) PCT Filed: Nov. 21, 2008
(86) PCT No.: PCT/KR08/06876

§ 371 Date: Jun. 28, 2010

(30) Foreign Application Priority Data
Nov. 22, 2007 (KR) 10-2007-0119930

(51) Int. Cl. A61K 31/337; A61P 43/00

(52) U.S. Cl. 514/449

(57) ABSTRACT

The present invention relates to a lyophilized composition with an improved reconstitution time comprising a taxoid and a method thereof. More specifically, the present invention relates to a lyophilized composition with an improved reconstitution time prepared by mixing and dissolving a taxoid; cyclodextrin (CD); a hydrophilic polymer selected from the group consisting of hydroxypropylmethyl CeIIuIoSe (HPMC), polyethylene glycol (PEG), and polyvinylpyrrolidone (PVP); and a bulking agent of saccharides in water for injection; and lyophilizing the mixture, and a method thereof.

### TECHNICAL FIELD

The present invention relates to a lyophilized composition with a reduced reconstitution time comprising a taxoid. The invention further relates to a method of preparation the same.

### BACKGROUND ART

Taxotere® (Sanofi-Aventis), an injection containing docetaxel consists of vial A containing a therapeutic ingredient and vial B containing 13% ethanol. It is prepared at the time of administration by injecting the content of vial B into vial A, mixing them by manually shaking it up and down for 45 seconds to obtain a premix solution (also named as an initial diluted solution) having 10 mg/mL of docetaxel concentration.

To avoid bubble generation in preparing the premix solution, shaking should be gently performed. Nevertheless, if bubbles are formed, however, the premix solution should be allowed to stand still until the bubbles disappear. The premix solution of the above Taxotere® is added in NaCl solution (0.9%, 250 mL) or dextrose solution (5%, 250 mL) to prepare a final concentration of 0.3-0.74 mg/mL and perfuse it into the blood vessels of a patient.

Abraxane® (Astra Zeneca), an injection containing paclitaxel, another kind of taxoid bound to albumin, is provided in the form of a lyophilized composition. Before administrating Abraxane®, 20 mL of saline solution for injection is added into a vial, the vial is allowed to stand still for 5 minutes, and then the vials is slowly shaken up and down for about 2-3 minutes to completely dissolve the lyophilized composition. If bubbles appear, the vial is allowed to stand still until the bubbles disappear and finally a solution having 5 mg/mL of paclitaxel is obtained.

The process of preparing a solution having an appropriate concentration of an active ingredient for the administration of a lyophilized composition as mentioned above, is called “reconstitution”. Short reconstitution time is preferable for both a member of medical center and patients. If the reconstitution time is too long, it will increase the preparation time thus making it difficult to administrate it to many patients at the same, which will eventually lower the competitiveness of the drug.

The present inventors developed a novel anticancer composition for injection comprising a taxoid having superior storage stability and dilution stability, improved solubility compared to those of conventional injections, without using a solubilizer such as polysorbate or ethanol which may cause adverse effects. Precisely, in order to solubilize and formulate the taxoid, hydroxypropyl β-cyclodextrin; a hydrophilic polymer such as hydroxypropylmethyl cellulose (HPMC), polyethylene glycol (PEG), or polyvinylpyrrolidone (PVP) were mixed and dissolved to in water for injection to prepare a lyophilized composition, and then an anticancer composition for injection having superior storage and dilution stability compared to those of conventional injections was obtained.

### DETAILED DESCRIPTION OF INVENTION

### Technical Problem

The present inventors added a bulking agent of saccharides such as dextrose or sorbitol to the lyophilized composition of a taxoid comprising hydroxypropyl β-cyclodextrin; a hydrophilic polymer such as hydroxypropylmethyl cellulose (HPMC), polyethylene glycol (PEG) or polyvinylpyrrolidone (PVP) in order to improve its physical properties while reducing the reconstitution time compared to those of conventional lyophilized compositions.

The present invention also relates to a lyophilized composition comprising a taxoid with improved physical properties and a method of its preparation.

### Technical Solution

The present invention relates to a lyophilized composition added with a bulking agent of saccharides to a composition comprising a water-insoluble taxoid; cyclodextrin; at least one hydrophilic polymer selected from the group consisting of hydroxypropylmethyl cellulose (HPMC), polyethylene glycol (PEG), and polyvinylpyrrolidone (PVP), and obtained a lyophilized composition comprising a taxoid improved in physical properties.

The present invention relates to a method of preparing a composition for injection comprising a taxoid with improved stability, which comprises:

1) dissolving a taxoid; cyclodextrin; a hydrophilic polymer such as hydroxypropylmethyl cellulose (HPMC), polyethylene glycol (PEG) or polyvinylpyrrolidone (PVP); and a bulking agent of saccharides in distilled water;

2) lyophilizing the mixture obtained in step 1).

### BEST MODE

The present invention relates to a lyophilized composition comprising a taxoid and a method of its preparation by mixing and dissolving a taxoid; cyclodextrin (CD); a hydrophilic polymer such as hydroxypropylmethyl cellulose (HPMC), polyethylene glycol (PEG) or polyvinylpyrrolidone (PVP); and a bulking agent in water for injection, and then lyophilizing the resultant. The lyophilized composition acquires porosity thereby improving the physical properties, and reduces the reconstitution time by using a diluent compared to the conventional lyophilized compositions.

The present invention relates to a method of preparing a lyophilized composition comprising a water-insoluble taxoid. In the method,
1) the first step is dissolving a water-insoluble taxoid, cyclodextrin, a hydrophilic polymer, and a bulking agent in water for injection. The water-insoluble taxoid is preferably a derivative as represented by the following Formula 1;

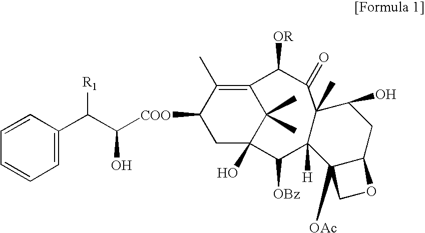

wherein R is a hydrogen atom or an acetyl group, R₁ is a tertiary-butoxycarbonylamino radical or a benzoylamino radical.

The taxoid represented by the Formula 1 is preferably docetaxel, wherein R is a hydrogen atom, R₁ is a tertiary-butoxycarbonylamino radical; or paclitaxel wherein R is an acetyl group and R₁ is a benzoylamino radical.

Further, the taxoid is in a free form or in the form of a pharmaceutically acceptable salt, anhydrous or hydrate thereof in the present invention. The quantity of the taxoid is preferably 0.2-50% (w/w), more preferably 0.2-20% (w/w), and most preferably 1.0-5.0% (w/w) in the lyophilized composition. If the content of the taxoid is low, a substantial amount of solution is required in the reconstitution. In contrast, if it is high, reconstitution time becomes long thus decreasing its commercial application.

Cyclodextrins are classified upon their properties and pore sizes as α-cyclodextrin, β-cyclodextrin, and γ-cyclodextrin. The available cyclodextrin in the invention includes derivatives of cyclodextrin, preferably β-cyclodextrin or derivatives thereof having 6.0-6.5 Å of diameter for each pore size, and more preferably hydroxypropyl β-cyclodextrin (HPBCD), an injection already in the commercial market and listed in the European Pharmacopoeia. Cyclodextrin is contained preferably 1-500 parts by weight relative to 1 part by weight of the taxoid, more preferably 5-200 parts by weight, and most preferably 5-100 parts by weight.

If cyclodextrin is used excessively, the resulting liquid composition becomes too viscous to filter it with 0.22 micrometer filter paper. On the contrary, if cyclodextrin is used too little, appropriate solubility and stability of the taxoid may not be obtained.

A degree of molecular substitution of hydroxypropyl (3-cyclodextrin (HPBCD) is preferably 0.2-1.0, and more preferably 0.4-1.0. If the degree of molecular substitution is too low, the solubility of HPBCD becomes low. In contrast, if it is too high, HPBCD becomes too viscous to handle.

The hydrophilic polymer used in the present invention increases the solubility and stability of the taxoid in the solution, and increases solubility of the taxoid by reacting with cyclodextrin.

Examples of the common hydrophilic polymers include polyethylene glycol (PEG), polyvinylpyrrolidinone (PVP), carboxymethylcellulose (CMC), hydroxypropylcellulose (HPC), hydroxymethylcellulose (HMC), hydroxyethylcellulose (HEC), hydroxypropylmethyl cellulose (HPMC), hydroxypropylethyl cellulose (HPEC), etc., and preferable hydrophilic polymers in the present invention are hydroxypropylmethyl cellulose (HPMC), polyethylene glycol (PEG) or polyvinyl pyrrolidone (PVP).

The viscosity of hydroxypropylmethyl cellulose (HPMC) is preferably 5-100,000 cps, and more preferably 5-4,000 cps. If the viscosity of hydroxypropylmethyl cellulose (HPMC) is too low, its solubility or stability of taxoid become remarkably poor. If the viscosity is too high, it is difficult to be treated and developed as an injection.

For the polyethylene glycol, there are various products with average molecular weight of 300-150,000. The preferable products of the polyethylene glycol are 300-600 in their average molecular weight, and more preferable products are 300, 400, and 600 which are acceptable as an injection.

Further, the K-value of polyvinylpyrrolidone is preferably in the range of 10-20. If it is below 10, the solubility and stability of taxoid becomes remarkably poor. Meanwhile, if it is over 20, viscosity increases and it is difficult to be used as an injection.

The content of the hydrophilic polymer is preferably 0.01-100 parts by weight, and more preferably 0.1-10.0 parts by weight relative to 1 part by weight of the taxoid. If it is below 0.01 parts by weight, the solubility and stability become remarkably low. On the contrary, if it is over 100, viscosity increases excessively and filtering, and cleaning thereafter becomes difficult.

Furthermore, it is preferable to use a bulking agent to form a channel that reduces the reconstitution time of the lyophilized composition. The bulking agent is preferably dextrose or sorbitol and the content thereof is preferably 1-50 parts by weight relative to 1 part by weight of the taxoid, more preferably 1-30 parts by weight, and most preferably 5-30 parts by weight. If the content of the bulking agent is less than 1 part by weight, the effect of bulking agent becomes less. Meanwhile, if the content is over 50 parts by weight, the lyophilization based on viscosity and solubility of the solution becomes difficult. Under the same condition as described above, dissolving a bulking agent of saccharides before lyophilization with other ingredients can reduce the reconstitution time compared to adding it in the reconstitution after lyophilization.

In fact, available bulking agents to be used in a lyophilized composition such as mannitol, lactose, sucrose, sodium chloride, trehalose, starch, hetastarch, glycine were not able to improve the physical properties of the lyophilized composition or reduce reconstitution time with a solvent.

There is no limit with regard to the solution used as a perfusate in the reconstitution according to the present invention, and preferably water for injection. The solution is prepared to have 1.5-30 mg/mL of taxoid concentration. If the concentration is lower than 1.5 mg/mL, the 1-batch productivity of the lyophilizator is lowered and the unit price is increased. If it is greater than 30 mg/mL, the solubility of the taxoid does not improve but the viscosity increases, thus making it difficult to conduct a commercial sterilization.

2) The second step is heating, stirring the mixture obtained in the above step 1) to acquire desired stability, and then, sterilizing by filtration, and lyophilizing the obtained composition, wherein the stirring is conducted at a temperature of 5-50° C., preferably 15-30° C. The resultant is frozen for the lyophilization at between −80 and −40° C. under the reduced pressure, and then a white or light yellow lyophilized composition is obtained.

The lyophilized composition obtained according to the present invention, achieves excellent stability which is not affected by temperature and humidity, thus it may be stored for a long time, easily prepared for a formulation for injection, and it is not decomposed by the influence of temperature and humidity during the manufacturing process. In addition, it can be safely administered to human body without a surfactant or an organic solvent causing hypersensitive side effects.

In order to formulate the composition obtained in the above step 2) into an injection, the lyophilized composition is diluted, and the diluent may be any solution which is usable as an injection, preferably water for injection, dextrose solution or saline.

The present invention may be further described with the Examples herein after but the invention is not limited to the same.

### Examples 1-4 and Comparative Examples 1-4

Docetaxel or paclitaxel, a hydrophilic polymer such as polyvinylpyrrolidone, hydroxypropyl β-cyclodextrin (HPBCD) and a bulking agent were weighed as in the Table 1 below, and homogeneously dissolved by stirring at room temperature. The resultant was filtered through 0.22 micrometer filter, cooled down at −45° C., and then lyophilized. Then, the lyophilized composition was completely dissolved in water for injection, and the reconstitution time for the concentration of docetaxel or paclitaxel to reach 5.0 mg/mL, was measured.

TABLE 1

| | Example | | | | Comparative Example | | | | |
|---|---|---|---|---|---|---|---|---|---|
| Category | 1 | 2 | 3 | 4 | 1 | 2 | 3 | 4 | 5 |
| Docetaxel | 800 | — | 800 | 800 | 800 | 800 | — | 800 | — |
| Anhydrous (mg) | | | | | | | | | |
| Paclitaxel (mg) | — | 800 | — | — | — | | 800 | — | 800 |
| HPbCD M = 0.6 | 24000 | 24000 | 32000 | 32000 | 32000 | 32000 | 32000 | 32000 | 32000 |
| (mg) | | | | | | | | | |
| PVP K-12 (mg) | 2400 | 2400 | 2400 | 2400 | 2400 | 2400 | 2400 | 2400 | 2400 |
| Dextrose (mg) | 5 | 20 | 10 | — | — | — | — | — | — |
| D-sorbitol (mg) | — | — | — | 10 | — | — | — | — | — |
| Mannitol (mg) | — | — | — | — | 10 | — | — | — | — |
| Lactose (mg) | — | — | — | — | — | 10 | — | — | — |
| NaCl (mg) | — | — | — | — | — | — | 2 | — | — |
| Reconstitution | 0.3 | 0.3 | 0.5 | 0.5 | 2 | 2 | 2 | 2 | 2 |
| Time (min) | | | | | | | | | |

### Experimental Example 2

### Measuring the Physical Properties of the Lyophilized Composition

The basic physical properties and the structures of the lyophilized compositions obtained in Examples 1-4 and Comparative Examples 1-4 were analyzed by measuring their XRD, TGA, DSC, and SEM. The results showed that there was no noticeable difference in their polymorphism and the structure of the lyophilized composition, but the reconstitution time was reduced because a bulking agent of saccharides facilitated to form a channel for a solvent to infiltrate into the lyophilized composition during the reconstitution.

### INDUSTRIAL APPLICABILITY

The present invention relates to a method improving the physical properties of a lyophilized composition comprising a taxoid. If the solid content in solution before lyophilization is equal to or more than 20%, the resultant lyophilized composition cannot formulate the channel and the reconstitution of the lyophilized composition consumes substantial time.

A bulking agent of saccharides or sugar alcohols improves the physical properties and stability of the solution before lyophilization, as well as it acquires porosity of the lyophilized composition in the reconstitution process and enables to reduce the reconstitution time.

Among the saccharides and sugar alcohols mentioned above, especially dextrose and D-sorbitol have excellent effects in improving physical properties of the lyophilized composition.

1. A lyophilized composition with a reduced reconstitution time comprising a taxoid; cyclodextrin; at least one hydrophilic polymer selected from the group consisting of hydroxypropylmethyl cellulose (HPMC), polyethylene glycol (PEG), and polyvinylpyrrolidone (PVP); and a bulking agent of saccharides.

2. The composition as claimed in claim 1, wherein said taxoid is represented by the following Formula 1; 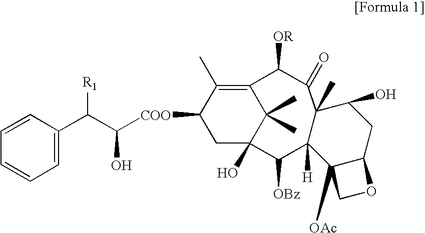
wherein R is a hydrogen atom or an acetyl group, R₁ is a tertiary-butoxy carbonylamino radical or a benzoylamino radical.

3. The composition as claimed in claim 2, wherein said taxoid is docetaxel represented by Formula 1, wherein R is a hydrogen atom, R₁ is a tertiary-butoxy carbonylamino radical.

4. The composition as claimed in claim 2, wherein said taxoid is paclitaxel represented by Formula 1, wherein R is an acetyl group and R₁ is a benzoylamino radical.

5. The composition as claimed in claim 2, wherein said taxoid is in a free form or in the form of a pharmaceutically acceptable salt, anhydrous or hydrate thereof.

6. A lyophilized composition with a reduced reconstitution time comprising a taxoid; cyclodextrin; a hydrophilic polymer selected from the group consisting of hydroxypropylmethyl cellulose (HPMC), polyethylene glycol (PEG), and polyvinylpyrrolidone (PVP); and a bulking agent of saccharides,
(a) which excludes a surfactant and an organic solvent;
(b) selectively comprises a pharmaceutically acceptable excipient, and
(c) the taxoid is contained 0.2-50 wt. %.

7. The lyophilized composition as claimed in claim 1, wherein the cyclodextrin is 1-500 parts by weight relative to 1 part by weight of the taxoid.

8. The composition as claimed in claim 7, wherein the cyclodextrin is 5-200 parts by weight relative to 1 part by weight of the taxoid.

9. The lyophilized composition as claimed in claim 1, wherein the hydrophilic polymers are 0.01-100 parts by weight relative to 1 part by weight of the taxoid.

10. The lyophilized composition as claimed in claim 1, wherein the cyclodextrin is β-cyclodextrin or derivatives thereof.

11. The lyophilized composition as claimed in claim 10, wherein the cyclodextrin is hydroxypropyl β-cyclodextrin.

12. The lyophilized composition as claimed in claim 1, wherein the bulking agent is 1-50 parts by weight relative to 1 part by weight of the taxoid.

13. A method of preparing a lyophilized composition comprising a taxoid with improved stability, which comprises:
1) mixing and dissolving a taxoid; cyclodextrin; a hydrophilic polymer selected from the group consisting of hydroxypropylmethyl cellulose (HPMC), polyethylene glycol (PEG), and polyvinylpyrrolidone (PVP); and a bulking agent of saccharides in water for injection; and
2) lyophilizing the mixture obtained in step 1).
